SEQUENCE LISTING (8 sequences)

SEQ ID NO: 1 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALRAQKEAISPPDAASAAPLRTITADTFRKLFRVYSNFLRGKLKLYTGEACRTGDR

SEQ ID NO: 2 - protein, 507 aa, Homo sapiens
MDQLRVARWPRVSPLCLLLAGAAWASSPSLPDPKFESKAALLASRGSEELLCFTQRLEDLVCFWEEAANSGMGFNYSFSYQLEGESRKSCRLHQAPTVRGSMRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRALRQKIWPGIPSPENEFEGLFTTHKGNFQLWLLQRDGCLWWSPSSPFPEDPPAHLEVLSERRWGVTQAGDAGAEDKGPLLEPVGSERAQDTYLVLDEWLLPRCPCSENLSGPGDSVDPATMDEGSETSSCPSDLASKPRPEGTSPSSFEYTILDPSSKLLCPRALPPELPPTPPHLKYLYLVVSDSGISTDYSSGGSQGVHGDSSDGPYSHPYENSLVPDTEPLRPSYVACS

SEQ ID NO: 3 - protein, 20 aa, Homo sapiens
GGTYSCHFGPLTWVCKPQGG

SEQ ID NO: 4 - protein, 20 aa, Homo sapiens
GGDYHCRMGPLTWVCKPLGG

SEQ ID NO: 5 - protein, 20 aa, Homo sapiens
GGVYACRMGPITWVCSPLGG

SEQ ID NO: 6 - protein, 20 aa, Homo sapiens
VGNYMCHFGPITWVCRPGGG

SEQ ID NO: 7 - protein, 20 aa, Homo sapiens
GGLYLCRFGPVTWDCGYKGG

SEQ ID NO: 8 - protein, 14 aa, Homo sapiens
GGCRIGPITWVCGG

(19) United States
(12) Patent Application Publication
(10) Pub. No.: US 2005/0004022 A1
(43) Pub. Date: Jan. 6, 2005

(54) Pharmaceutical compositions for treating painful neuropathy and methods of treating same

(76) Inventors: Murat Digicaylioglu, San Diego, CA (US); Stuart A. Lipton, Rancho Santa Fe, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/810,362

(22) Filed: Mar. 25, 2004

Related U.S. Application Data
(60) Provisional application No. 60/457,532, filed on Mar. 25, 2003.

(51) Int. Cl. A61K 38/18; A61K 38/17

(52) U.S. Cl. 514/12

(57) ABSTRACT

The present invention provides novel methods and compositions for the treatment and prevention of HIV-associated dementia (HAD), minor cognitive/motor disorder (MCMD), neuropathic pain associated with HAD, or neuropathic pain from other causes.

### RELATED APPLICATIONS

This application claims priority to U.S. Ser. No. 60/457,532 filed Mar. 25, 2003 the contents of which is incorporated herein by reference in its entirety.

### FIELD OF THE INVENTION

This invention relates to compositions and methods comprising erythropoietin (EPO) for treating painful neuropathy.

### BACKGROUND OF THE INVENTION

One symptom of human immunodeficiency virus (HIV) is a syndrome of cognitive and motor dysfunction that has been designated HIV-associated dementia (HAD). Although in the era of highly active antiretroviral therapy (HAART), a milder form of neurologic dysfunction, termed minor cognitive/motor disorder (MCMD), may have become more prevalent than frank dementia. HAD and MCMD remain significant independent risk factors for acquired immunodeficiency syndrome (AIDS) mortality (Kaul, M., et al., Nature 410:988-994 (2001), and Power, C., et al. Can. J. Neurol. Sci. 29:19-32, (2002)). Despite improvements in control of peripheral viral replication and the treatment of opportunistic infections, HAART fails to provide complete protection from the development of HAD.

### SUMMARY OF THE INVENTION

The invention features compositions and methods for inhibiting neuronal cell death such as that associated with an infectious disease, e.g., HIV-1 infection. The method is carried out by contacting an HIV-1-exposed neuronal cell with an apoptosis-inhibitory amount of EPO polypeptide. The EPO is administered either before or after exposure of the neuronal cell to HIV-1 or gp120. The EPO is also administered either before or after detection of neuronal symptoms. Preferably, the neuronal cell is contacted with the EPO polypeptide prior to commencement of gp120-induced apoptosis. Neuronal cell death is reduced in the presence of EPO compared to in its absence. For example, neuronal cell death is reduced by 10%, 50%, 100%, 200%, or more, in the presence of or following contacting the cell with EPO compared to in its absence. The method optionally includes the step of contacting the neuronal cell or neuronal tissue with an EPO receptor polypeptide.

Also within the invention is a method of inhibiting neuronal cell death, by preferentially contacting a neuronal tissue with an apoptosis-inhibitory amount of an EPO polypeptide. For example, the EPO is administered intranasally or intrathecally to minimize exposure of non-neuronal tissues to the administered EPO. Optionally, an EPO receptor polypeptide is administered.

A method of reducing a symptom a neurological disorder an infectious disease-associated neuropathy or chronic disease associated neuropathy (e.g., diabetes-related neuropathy) is carried out by identifying an individual suffering from an infectious disease (e.g., HIV-1) or a chronic disease with neurological symptoms, and administering to the individual a therapeutically effective amount of erythropoietin. Symptoms such as pain and cognitive dysfunction such as minor or serious dementia are reduced following administration of an EPO composition. For example, the subject is male or a non-lactating female.

A therapeutically-effective amount of EPO is an amount that reduces a symptom of a neurological disorder. EPO is preferably administered in an amount that reduces gp120-induced apoptotic death of neuronal cells, CXCR4-activation, astrocytosis, infiltration of macrophages, increased number of microglia, multinucleated giant cells, myelin pallor, dendritic and synaptic damage, apoptosis leading to frank loss of neurons, accumulation of macrophages/microglia, gp41-, gp160-, Tat-, Nef-, Rev-, or Vpr-induced apoptotic death of neuronal cells, Ca2+ overload, activation of p38 mitogen-activated protein kinase (MAPK), release of cytochrome c from mitochondria, caspase activation, free-radical formation, lipid peroxidation, and chromatin condensation. For example, a therapeutically effective amount of erythropoietin is between about 1 U/kg/day and 2000 U/kg/day and a therapeutically effective amount of soluble erythropoietin receptor is between about 1 U/kg/day and 2000 U/kg/day. Optionally, IGF-1 is co-administered or administered in conjunction with EPO (e.g., within minutes, 1-12 hours, or 1-7 days before or after EPO administration. A therapeutically effective amount of insulin-like growth factor-I is between about 1 U/kg/day and 2000 U/kg/day. The compositions are administered intranasally, intrathecally, or intravenously. The soluble erythropoietin receptor is present in an amount that increases the stability of erythropoietin when both are administered to a patient.

Also within the invention are pharmaceutical compositions containing erythropoietin, a soluble erythropoietin receptor, and a pharmaceutically acceptable carrier and kits containing in one or more containers, the pharmaceutical composition(s) listed above. The compositions described herein are purified. For example, recombinant proteins are expressed in Chinese hamster ovary (CHO) cells, or using other recombinant methods and isolated rom cultured cells using methods known in the art. By purified or isolated is meant that the desired protein or polypeptide is 85% of the composition by weight (w/w). Preferably, the desired EPO polypeptide is at least 90, 95, 98, 99, or 100% of the composition by weight (w/w).

### DETAILED DESCRIPTION OF THE INVENTION

Erythropoietin (EPO) is administered with or without simultaneous administration of soluble EPO Receptor (which increases the short half-life of EPO) for treating HIV-associated dementia (HAD), minor cognitive/motor disorder (MCMD), neuropathic pain associated with HAD, or neuropathic pain from other causes. EPO protects neurons from injury and apoptosis due to exposure to toxins related to HIV-1 including the envelope glycoprotein gp120.

HIV-1 envelope glycoprotein gp120 induces neuronal injury and apoptosis, which contributes to HIV-associated dementia (Kaul, et al., Nature 410:988-994 (2001)). EPO is a neuroprotective cytokine produced in the brain (Digicaylioglu & Lipton, Nature 412:641-647 (2001)), but its effect on gp120-induced neuronal damage has not been previously assessed.

Erythropoietin (EPO) is the principal growth factor that induces proliferation and differentiation of erythroid progenitor cells and is a member of the cytokine family that includes interleukins 2 through 7, G-CSF, GM-CSF, TPO, growth hormone and leptin (Koury and Bondurant, Transfusion 30: 673-674 (1992)).

Binding of EPO to its receptor triggers signal transduction by ligand-mediated receptor dimerization on the cell surface. Point mutations that introduce cysteine residues into the membrane proximal part of the extracellular domain of the EPO receptor, and which result in disulfide-linked receptor dimers on the cell surface, are constitutively active. Such receptors lead to cell proliferation of EPO-dependent cell lines and other biological effects of EPO in the absence of the hormone (Yoshimura et al., Nature 348: 647-649 (1990); Watowich et al., Proc. Natl. Acad. Sci. , USA 89: 2140-2144 (1992); and Watowich et al., Mol. Cell. Biol. 14: 3539-3549 (1994) ). Expression of these constitutive EPO receptors in mice results in erythroleukemia through unregulated activation of the signaling pathway (Longmore and Lodish, Cell 67:1089-1102 (1991); Longmore et al., Mol. Cell. Biol. 14: 2266-2277 (1994)). EPO receptor activation has been shown to follow a sequential dimerization mechanism, with binding to a high affinity site 1 on EPO preceding binding of the second receptor to a lower affinity site 2 (Matthews et al., Proc. Nati. Acad. Sci., USA 93:9471-9476 (1996)).

As used herein, the term “erythropoietin” is synonymous with “EPO” and means a polypeptide that has substantially the amino acid sequence of naturally occurring human EPO (SEQ ID NO:1) or a homolog thereof. EPOs useful in the invention include human and other primate EPOs, mammalian EPOs such as bovine, porcine, murine and rat homologs and other vertebrate homologs such as Danio rerio homologs. Thus, the term EPO encompasses species homologs, alternatively spliced forms, isotype and glycosylation variants and precursors-of the mature human EPO sequence (SEQ ID NO:1) shown in below in Table 1.

TABLE 1

Amino acid sequence of mature human
erythropoietin

| | |
|---|---|
| APPRLICDSRVLERYLLEAKEAENITTGCAEHCSLNE | (SEQ ID NO:1) |
| NITVPDTKVNFYAWKRMEVGQQAVEVWQGLALLSEAV | |
| LRGQALLVNSSQPWEPLQLHVDKAVSGLRSLTTLLRA | |
| LRAQKEAISPPDAASAAPLRTITADTFRKLFRVYSNF | |
| LRGKLKLYTGEACRTGDR | |

An EPO generally has an amino acid sequence with at least about 80% amino acid identity to the sequence of naturally occurring, mature human EPO (SEQ ID NO:1) and can have, for example, 90% or 95% or more amino acid identity with SEQ ID NO:1. Sequence identity is determined using BLASTP at the NCBI website using the standard parameters.

Native erythropoietin is heavily glycosylated, and EPO prepared from Chinese hamster ovary (CHO) cells has three N-linked and one O-linked glycosylation sites with the average carbohydrate content being about 40%. In native EPO, carbohydrate plays an important role in stability, biosynthesis, apical secretion and biological activity. In particular, glycosylation appears to increase both conformational stability and solubility of EPO, although conformation is not affected. Thus, an EPO analog also can be a form of EPO that is hyper-glycosylated compared to native human EPO. Such analogs are known in the art and include, without limitation, Darbepoietin.

A variety of forms of erythropoietin with varying glycosylation patterns are available commercially, including but not limited to, EPOGEN (Amgen; Thousand Oaks, Calif.); EPOGIN (Chugai Pharmaceuticals; Tokyo, Japan); EPOMAX (Elanex; Bothell, Wash.); EPREX (Janssen-Cilag; Beerse, Belgium); NEORECORMON and RECORMON (Roche; Basel, Switzerland) and PROCRIT (Ortho Biotech; Raritan, N.J.). Various forms of EPO also are available generically as EPOETIN ALFA, EPOETIN BETA and EPOETIN OMEGA. Thus, it is understood that an EPO useful in the invention can be obtained commercially or by a variety of well known methods, including, without limitation, purification from a natural source, recombinant expression, or-peptide or chemical synthesis A method of the invention can be practiced, if desired, with an “EPO analog” As used herein, the term “EPO analog” means a molecule that induces or enhances the expression, activity or intracellular signaling of the erythropoietin receptor and that, in combination with an insulin-like growth factor, produces a synergistic acute neuroprotective effect in neurons. Such an analog can be, without limitation, a protein, peptide, peptidomimetic, small molecule, ribozyme, nucleic acid molecule, oligonucleotide, oligosaccharide, cell, phage or virus, or a combination thereof. As described further below, EPO analogs useful in the invention encompass, yet are not limited to, erythropoietin mimetic peptides (EMPs); cyclic molecules such as cyclic peptides or peptidomimetics; dimeric and oligomeric EPO analogs; analogs with increased plasma half-life; anti-EPO receptor antibodies; small molecule drugs that induce EPO receptor dimerization; hyper-glycosylated forms of EPO; EPO-encoding nucleic acid molecules; and constitutive forms of the EPO receptor. It is understood that the term EPO analog encompasses active fragments of EPO, which are described hereinabove.

Soluble EPO receptor can be administered with EPO to a patient to increase the half-life of EPO in the patient. An example of an amino acid sequence of an EPO receptor is shown below in Table 2.

TABLE 2
Amino acid sequence of human EPO receptor

| | | |
|---|---|---|
| 1 | mdqlrvarwp rvsplcllla gaawasspsl pdpkfeskaa llasrgseel lcftqrledl | (SEQ ID NO:2) |
| 61 | vcfweeaans gmgfnysfsy qlegesrksc rlhqaptvrg smrfwcslpt adtssfvple | |
| 121 | lqvteasgsp ryhriihine vvlldapagl larraeegsh vvlrwlpppg apmtthirye | |
| 181 | vdvsagnrag gtqrvevleg rtecvlsnlr ggtrytfavr armaepsfsg fwsawsepas | |
| 241 | lltasdldpl iltlslilvl isllltvlal lshrralrqk iwpgipspen efeglftthk | |
| 301 | gnfqlwllqr dgclwwspss pfpedppahl evlserrwgv tqagdagaed kgpllepvgs | |
| 361 | eraqdtylvl dewllprcpc senlsgpgds vdpatmdegs etsscpsdla skprpegtsp | |
| 421 | ssfeytildp sskllcpral ppelpptpph lkylylvvsd sgistdyssg gsqgvhgdss | |
| 481 | dgpyshpyen slvpdteplr psyvacs | |

Recombinant human EPO forms a dimer upon extensive heating, based on intermolecular disulfide bond formation involving cysteines-7 and -161. Thus, oligomeric forms of erythropoietin, as well as oligomeric EPO fragments and analogs thereof, can be useful in the invention. See, in general, DePaolis et al. , J. Pharm. Sci. 84: 1280-1284 (1995), and Derby et al. , Int. J. Peptide Protein Res. 47: 201-208 (1996).

Oligomeric forms of EPO or active fragments or analogs thereof useful in the invention include dimers and trimers as well as higher multimeric forms. An oligomeric form of EPO can include two or more, three or more, four or more, five or more, six or more, seven or more, eight or more, nine or more, ten or more, fifteen or more, twenty or more, 50 or more, 100 or more, 200 or more, 500 or more, or 1000 or more copies, of EPO or an active fragment or analog thereof. As examples, chemical cross-linking, synthetic peptide chemistry, phage display and conjugation of biotin-tagged EPO with streptavidin can be useful in generating oligomeric EPO analogs.

Dimeric and trimeric EPO analogs can be formed using heterobifunctional crosslinking reagents, for example, by chemically modifying a first pool of erythropoietin monomers to contain free sulfhydryl residues and mixing this pool with a second pool containing maleimido groups; the oligomeric EPO subsequently can be purified, for example, by-size exclusion HPLC.

Native human erythropoietin has a relatively-short plasma half-life of about 4 to 13 hours, while EPO analogs with a larger molecular size can have a reduced rate of clearance and, therefore, increased plasma survival and in vivo biological activity. Thus, higher molecular weight EPO analogs including oligomeric forms of EPO, or active fragments or analogs thereof, can exhibit an increased plasma half-life as compared to the half-life of native monomeric human EPO (Sytkowski et-al., Proc. Natl. Acad. Sci. USA 95: 1184-1188(1998)). An oligomeric form of EPO can have, for example, a half-life of at least 15, 18, 21, 24, 48, 72 or 96 hours. One skilled in the art recognizes that, if desired, soluble EPO receptor can be included to increase the half-life of native erythropoietin, or an active fragment or analog thereof, and, therefore, therapeutic value.

Many forms of erythropoietin, as well as active-fragments and analogs thereof, can be useful in the methods of the invention. Neuronal cells are contacted with EPO or an active fragment thereof, for example, with human EPO or an active fragment thereof. In another embodiment, neuronal cells are contacted with an EPO analog, which can be, without limitation, a peptide, peptidomimetic, small molecule or nucleic acid EPO analog. A preferred EPO analog includes the amino acid sequence of

| | |
|---|---|
| GGTYSCHFGPLTWVCKPQGG; | (SEQ ID NO:3) |
| GGDYHCRMGPLTWVCKPLGG; | (SEQ ID NO:4) |
| GGVYACRMGPITWVCSPLGG; | (SEQ ID NO:5) |
| VGNYMCHFGPITWVCRPGGG; | (SEQ ID NO:6) |
| GGLYLCRFGPVTWDCGYKGG; | (SEQ ID NO:7) |
| or | |
| GGCRIGPITWVCGG. | (SEQ ID NO:8) |

EPO, or an active fragment or analog thereof, preferably has at least 10-fold higher affinity for the EPO receptor than native human EPO. The EPO protein or an active fragment is oligomeric, for example, dimeric. As an example, such a dimeric form of EPO is a dimer in which each monomer contains the amino acid sequence

| | |
|---|---|
| GGTYSCHFGPLTWVCKPQGG. | (SEQ ID NO:3) |

Neuropathology of HIV Infection

HAD is often accompanied by certain neuropathological findings, such as astrocytosis, infiltration of macrophages, increased number of microglia, multinucleated giant cells, myelin pallor, dendritic and synaptic damage, and apoptosis leading to the frank loss of neurons. The accumulation of macrophages/microglia correlates with the severity of HAD HIV-1 infected or immune-stimulated macrophages/microglia produce neurotoxins. Importantly, neurons are not productively infected by HIV-1 and astrocytes only rarely, primarily in pediatric cases.

Interestingly, even in the absence of intact virus, the HIV proteins gp120, gp41, gp160, Tat, Nef, Rev, and Vpr have been reported to initiate neuronal damage, at least in vitro, and in some cases in vivo in animal models. Intracerebroventricular injection of gp120 causes brain injury in vivo in rodents and a transgenic mouse model expressing gp120 develops many neuropathological features observed in postmortem brain specimens from HAD patients.

Chemokine Receptors in HAD

Infection of macrophages and lymphocytes by HIV-1 occurs after binding of the viral envelope protein gp120 to one of several possible chemokine receptors in conjunction with CD4. Macrophages and microglia are primarily infected via the β-chemokine receptor CCR5 or CCR3, but the β-chemokine receptor CXCR4 may also be involved. The HIV coreceptors CCR5 and CXCR4, among other chemokine receptors, are also present on neurons and astrocytes. CXCR4 is directly involved in HIV-associated neuronal damage, whereas CCR5 may additionally serve a protective role.

In cerebrocortical neurons and neuronal cell lines, picomolar concentrations of HIV-1 gp120, as well as intact virus, can induce neuronal death via CXCR4 receptors. In mixed neuronal/glial cerebrocortical cultures that mimic the cellular composition of the intact brain, this apoptotic death appears to be mediated predominantly via the release of microglial toxins, rather than by direct neuronal damage. However, nanomolar concentrations of (stromal cell derived factor) SDF-1 a interacting with CXCR4 can induce apoptotic death of neurons in the absence of microglia, suggesting a possible direct interaction with neurons while interaction with astrocytes can also occur. In contrast to these findings, somewhat higher concentrations of SDF-1α have been reported to provide neuroprotection from X4-preferring gp120-induced damage of isolated hippocampal neurons. However, the results obtained on isolated neurons may be different from those observed in mixed neuronal/glial cultures because the non-neuronal cells are known to modify the involved death pathways.

The role of chemokine receptors in the neurotoxicity of gp120 using mixed neuronal/glial cerebrocortical cultures from rat and mouse was investigated. gp120 from CXCR4 (X4)-preferring as well as CCR5 (R5)-preferring and dual-tropic HIV-1 strains all were able to trigger neuronal death. β-chemokines macrophage inhibitory protein (MIP)-1β and RANTES abrogated gp120 neurotoxicity. Interestingly, although HIV-1 gp120 of one X4-preferring strain lacked neurotoxicity in CXCR4-deficient cerebrocortical cultures, gp120 of another X4-classified strain retained some residual ability to induce neuronal death, as the dual-tropic gp120_{SF2} did. Surprisingly, gp120_{SF2} showed even greater neurotoxicity in CCR5 knockout cultures, compared to wild-type or CXCR4-deficient cultures. These findings are consistent with a primarily neurotoxic effect of CXCR4 activation by gp120. In contrast, activity of CCR5 is at least in part neuroprotoctive. Nonetheless, gp120 from R5-preferring HIV-1 can also induce neuronal death. However, it is important to bear in mind that the classification of HIV-1 as R5- or X4-preferring is largely based on the virus' ability to use certain receptors for infection of a target cell rather than activation or induction of a death signaling pathway. Consequently, this classification is only valid for an interaction of the HIV envelope that leads to infection. Therefore, in order to understand the mechanism(s) of HAD, gp120s from HIV-1s classified as preferring a certain chemokine receptor for infection may have to be reassessed for their ability to also interact with other receptors that trigger activation and downstream intracellular signaling. In particular, gp120s of R5-preferring macrophagetropic strains may need to be tested for their ability to initiate cellular signaling via CXCR4.

Because inhibition of microglial activation is sufficient to prevent neuronal death after gp120 exposure, at least in vitro, it seems likely that stimulation of CXCR4 in macrophages/microglia is a prerequisite for the neurotoxicity of gp120. In contrast, SDF-1 might directly activate CXCR4 in astrocytes and neurons to trigger neuronal death, for example, by reversing glutamate uptake in astrocytes.

HIV-1 in the Brain and NMDA Receptor Activation

Analysis of specimens from AIDS patients as well as in vivo and in vitro experiments indicate that HIV-1 infection creates excitotoxic conditions in the CNS. Macrophages and microglia play a crucial role because they are the predominant cells productively infected with HIV-1 in the brain. Moreover, HIV-1 infected or gp120-stimulated mononuclear phagocytes have been shown to release neurotoxins that directly stimulate the NMDA receptor, including quinolinic acid, cysteine, platelet-activating factor (PAP), and a low-molecular-weight compound designated NTox.

Additionally, HIV-infected or -activated macrophages/microglia and possibly astrocytes produce inflammatory mediators, including tumor necrosis factor (TNF)-α, arachidonic acid metabolites, free radicals (reactive oxygen species [ROS]) and nitric oxide [NO]) and extracellular matrix-degrading enzymes, such as matrix metalloproteinases (MMPs), that may indirectly contribute to excitotoxic neuronal damage. Along these lines, gp120 has been found to aggravate excitotoxic conditions by impairing astrocyte uptake of glutamate via arachidonic acid that is released from activated macrophages/microglia. SDF-1, TNF-α, and prostaglandins also can stimulate a Ca²⁺ dependent release of glutamate by astrocytes.

HIV-1 infection and its associated neurological dysfunction involved both chomokine receptor- and NMDA receptor-mediated excitotoxicity. Chemokine receptors are involved at different levels, first In HIV infection, and further, in the response to chemokines, which may be produced as a consequence of viral infection. For example, monocyte chemoattractant protein (MCP)-1, MIP-1α, MIP-β, RANTES, and SDF-1 are all likely to interfere, directly and/or indirectly, with the physiological functions of neurons, astrocytes, and microglia. NMDA receptors respond to excitatory agents, such as neurotransmitters and neurotoxins, but G protein coupled chemokine receptors might also influence their activity, and vice versa. A β-chemokine, RANTES, can diminish neuronal damage induced by excessive NMDA receptor stimulation. In turn, excitotoxic stimulation can enhance expression of CCR5.

Downstream Pathways from NMDA Receptors

If excessive stimulation of the NMDA receptor occurs and the initial excitotoxic insult is fulminant, the cells die early from loss of ionic homeostasis, leading to acute swelling and lysis (necrosis). If the insult is more mild, as it appears to be the case with HAD, neurons enter a programmed death pathway known as apoptosis. Neuronal apoptosis after excitotoxic insult involves Ca²⁺ overload, activation of p38 mitogen-activated protein kinase (MAPK), release of cytochrome c from mitochondria, caspase activation, free-radical formation, lipid peroxidation, and chromatin condensation. The p38 MAPK phosphorylates and activates transcription factors, including myocyte enhancer factor 2 (MEF2). Interestingly, cleavage of MEF2 by caspases can also contribute to neuronal apoptosis. Caspase-3 and -7 generate truncated MEF2 molecules that lack transcriptional activity, but still bind to DNA. Thus, the MEF2 fragments apparently compete with uncleaved MEF2 and consequently interfere with survival-promoting gene transcription in neurons.

Antibody-mediated neutralization of TNF-α or inhibition of its downstream effector caspase-8 also prevents the neurotoxicity of HIV gp120 in cultured cerebrocortical neurons. Caspase-8 activation can trigger caspase-3 activity, leading to apoptosis. At least in vitro, proteins of the Bcl-2 family possess the potential to abrogate neuronal damage subsequent to HIV-1 infection. An apparent inability of neurons to up-regulate Bcl-2 or Bcl-xL in response to the excitotoxicity generated by HIV infection might contribute to neuronal vulnerability.

The scaffolding protein PSD-95 (poatsynaptic density-95) links the NMDA receptor operated ion channel with neuronal nitric oxide synthase (nNOS), a Ca²⁺ activated enzyme, and thus brings nNOS into close proximity to Ca²⁺. Excessive intracellular Ca²⁺ overstimulates nNOS and protein kinase cascades, causing the generation of cytotoxic free radicals, including ROS, NO, and peroxynitrite (ONOO⁻). ROS and NO can form the highly cytotoxic ONOO⁻. Data from a study in postmortem brain specimens from HIV patients indicated that the expression of immunologic NOS (iNOS) and gp41 correlate with the occurrence and severity of HAD. Furthermore, the neurotoxic effect of gp120 is, at least in vitro, also dependent on NOS (possibly both nNOS and iNOS).

In addition to the intracellular effects of NO, a potential extracellular proteolytic pathway to neuronal injury that is mediated by nitrosylation and subsequent activation of MMP-9 has recently been identified. Proteolytically active MMP-9 incites neuronal death, presumably by disrupting the interaction of cellular adhesion factors and extracellular matrix. The expression and activation of MMPs, including MMP-2 and MMP-9, is increased in HIV infected macrophages and also in postmortem brain specimens from AIDS patients when compared with uninfected controls.

Activation of MMPs may accompany excitotoxic brain injury and NOS activity. For example, nitrosylation of MMP-9 resulted in its activation. Subsequently, the in vitro effects of NO-activated MMP-9 on neurons in cerebrocortical cultures was investigated. For these experiments, recombinant (R)-proMMP-9 was preactivated with the endogenous NO donor S-nitrosocysteine (SNOC) before addition to the cultures. Neuronal apoptosis was assessed 18 h later. Because NO had already been released from SNOC by the time the cultures were incubated with the activated MMP, direct release of NO from SNOC or the formation of peroxynitrite due to the release of NO from SNOC and subsequent reaction with suporoxide anion (O⁻₂) could not trigger neuronal death. However, NO-activated MMP-9 significantly increased the apoptosis of neurons, whereas treatment with inactive proMMP-9 or the MMP inhibitor GM6001 blocked the neuronal cell death. In addition, many neurons came up off the dish after exposure to NO-activated MMP-9. These results indicate that inactive proMMP-9 protein does not have a deleterious effect on neurons. However, NO-triggered activation converts MMP-9 into a neurotoxin.

Therapeutic Approaches for Treatment of HAD

A truly effective pharmacotherapy for HAD has yet to be developed. Accumulating evidence regarding the pathogenesis of HAD indicates that several potential therapeutic strategies are applicable in addition to antiretrovirals (ARVs). The clinically tolerated NMDA receptor antagonist memantine is among the agents under consideration. Others include β-chemokines; chemokine and cytokine receptor antagonists, e.g., against CXCR4 and CCR5 activation; inhibitors of MMPs, p38 MAPK, or caspases; and antioxidants.

Chemokine receptors mediate HIV-1 infection and specific chemokines block infection in vivo. Additionally, elevated concentrations of β-chemokines in the CSF of HIV patients correlate with relatively better neuropsychological performance.

Concerning NMDA receptor antagonists, it was found that memantine is an uncompetitive, open-channel blocker of the NMDA receptor associated ion channel. Memantine blocks the NMDA receptor-operated channel only when it is open for pathological periods of time. Conversely, memantine has little effect during normal neurotransmission, when there is less NMDA receptor dependent channel activity. In fact, memantine was shown to hold promise for HAD in a recent phase II clinical trial, and demonstrated a clear positive effect in a phase m trial of Alzheimer's disease patients with moderate-to-severe dementia.

Nitroglycerin, which produces a NO-related nitrosonium ion (NO⁺), acts, at least in part, at redox modulatory sites on the NMDA receptor/channel complex to diminish receptor activity and consequent neuronal damage due to excessive Ca²⁺ influx.

Schedule of Administration

The compositions of the invention are administered in any suitable fashion to obtain the desired treatment of HIV-associated dementia (HAD), minor cognitive/motor disorder (MCMD), neuropathic pain associated with HAD, or neuropathic pain from other causes.

The present invention provides a more effective method of treatment for HAD, and pharmaceutical compositions for treating HAD, which may be used in such methods.

The invention further relates to kits for treating patients having HAD, comprising a therapeutically effective dose of EPO for treating or at least partially alleviating the symptoms of the condition, and instructions for its use.

The present invention is suitable for the reduction of HAD symptoms. These HAD symptoms include symptoms associated with, or arising from, HAD, and include neuropathic pain.

To evaluate whether a patient is benefiting from the (treatment), one examines the patient's symptoms in a quantitative way, e.g., by decrease in neuropathic pain. In a successful treatment, the patient status will have improved (i.e., decrease in the symptoms of neuropathic pain.

Symptoms of dementia include loss of memory, problem-solving ability, decision making ability, judgment, ability to orient oneself in space, and the ability to put together simple sentences and communicate with words. It is often also associated with personality change. Symptoms of neuropathy include numbness, pain, weakness, and loss of position sense.

Neuropathy is diagnosesd by measuring nerve conduction velocity (NCV) or electromyography (EMG). Nerve conduction velocity studies record the speed at which impulses travel through nerves and measure electrical responses. EMG records electrical activity in muscle tissue and is used to distinguish neuropathy from muscle disease (myopathy).

Dementia is diagnosed through observation of evidence of (1) erosion of recent and remote memory and (2) impairment of one or more of the following functions: misuse of words or inability to remember and use words correctly (i.e., aphasia); impairment of the ability to perform motor activities even though physical ability remains intact (i.e., apraxia); impairment of the ability to recognize objects, even though sensory function is intact (i.e., agnosia); or impairment of the ability to plan, organize, think abstractly.

As for every drug, the dosage is an important part of the success of the treatment and the health of the patient. In every case, in the specified range, the physician has to determine the best dosage for a given patient, according to his sex, age, weight, pathological state and other parameters.

The pharmaceutical compositions of the present invention contain a therapeutically effective amount of the active agents. The amount of the compound will depend on the patient being treated. The patient's weight, severity of illness, manner of administration and judgment of the prescribing physician should be taken into account in deciding the proper amount. The determination of a therapeutically effective amount of EPO is well within the capabilities of one with skill in the art.

In some cases, it may be necessary to use dosages outside of the ranges stated in pharmaceutical packaging insert to treat a patient. Those cases will be apparent to the prescribing physician. Where it is necessary, a physician will also know how and when to interrupt, adjust or terminate treatment in conjunction with a response of a particular patient.

Formulation and Administration

The compounds of the present invention are administered in a suitably formulated dosage form. Compounds are administered to a patient in the form of a pharmaceutically acceptable salt or in a pharmaceutical composition. A compound that is administered in a pharmaceutical composition is mixed with a suitable carrier or excipient such that a therapeutically effective amount is present in the composition. The term “therapeutically effective amount” refers to an amount of the compound that is necessary to achieve a desired endpoint (e.g., decreasing symptoms associated with HAD).

A variety of preparations can be used to formulate pharmaceutical compositions containing EPO, including solid, semi solid, liquid and gaseous forms. Techniques for formulation and administration may be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations. The formulations can be administered in either a local or systemic manner or in a depot or sustained release fashion. Administration of the composition can be performed in a variety of ways. In a preferred embodiment, the route of administration is intranasal or intravenous. In other embodiments, the route is oral, buccal, rectal, parenteral, intraperitoneal, intradermal, transdermal, and intratracheal means can be used. The compositions of the invention may be administered in combination with a variety of pharmaceutical excipients, including stabilizing agents, carriers and/or encapsulation formulations as described herein.

The preparation of pharmaceutical or pharmacological compositions will be known to those of skill in the art in light of the present disclosure. Typically, such compositions may be prepared as solid forms; as tablets or other solids for oral administration; as time release capsules.

For human administration, preparations should meet sterility CMC manufacturing standards as required by FDA.

Administration of compounds alone or in combination therapies are anticipated to be intravenous or intranasal delivery (solid or liquid). A particularly convenient frequency for the administration of the compounds of the invention is once a day or twice a day.

Upon formulation, therapeutics will be administered in a manner compatible with the dosage formulation, and in such amount as is pharmacologically effective. The formulations are easily administered in a variety of dosage forms, such as intravenous or intranasal described. In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically used. Suitable regimes for administration are also variable, but would be typified by initially administering the compound and monitoring the results and then giving further controlled doses at further intervals. The compounds and combination therapies of the invention can be formulated by dissolving, suspending or emulsifying in an aqueous or nonaqueous solvent. Vegetable (e.g., sesame oil) or similar oils, synthetic aliphatic acid glycerides, esters of higher aliphatic acids and propylene glycol are examples of nonaqueous solvents. Aqueous solutions such as Hank's solution, Ringer's solution or physiological saline buffer can also be used.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Oral preparations can be formulated through combination with pharmaceutically acceptable carriers that are well known in the art. The carriers enable the compound to be formulated, for example, as a tablet, pill, capsule, solution, suspension, sustained release formulation; powder, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, non gluten wheat starch, potato starch, gelatin, gum tragacanth, methyl cellulose, hydroxypropylmethylcellulose, sodium carboxymethylcellulose and polyvinylpyrrolidone (PVP). Oral formulations include such normally employed excipients as, for example, pharmaceutical grades of mannitol, lactose, starch, magnesium stearate, sodium saccharine, cellulose, magnesium carbonate and the like.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

The compositions of the present invention can also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The subject treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

The pharmaceutical compositions of the invention are used to treat HAD. Also treated by the pharmaceutical compositions of the invention are symptoms arising from HAD. Symptoms associated with, or arising from, HAD, include neuropathic pain.

The neuroprotective action of EPO to reduce gp120-induced neurotoxicity was evaluated as described below.

### EXAMPLES

### Example 1

Design/Methods: Neuronal/glial cortical cultures were mixed containing 30-40% neurons. The cultures were prepared from embryonic day 16-17 rats and used 17 days after plating. Cultures were exposed to 200 pM gp120 for 24 hours in the presence or absence of EPO (10 U/ml). Apoptotic neurons were identified by labeling with MAP-2, a neuron-specific marker, and TUNEL staining plus morphology. Intracellular signaling pathways were identified by immunoblot and immunocytochemistry for Akt and GSK3. Transduction with a dominant-interfering form of Akt was achieved with an adenoviral construct.

Results: HIV/gp120 increased neuronal apoptosis compared to control (506% versus 114%). Preincubation for 3 hours with EPO decreased apoptosis to 387%. Next, cellular signaling involved in EPO neuroprotection from gp120 was investigated. Exposure to gp120 resulted in dephosphorylation of GSK3. The neuroprotective effect of EPO was largely blocked by inhibition of Akt via transduction with a dominant interfering Akt mutant, preventing phosphorylation of GSK3.

### Equivalents

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby fully incorporated by reference. The appropriate components, processes, and methods of those patents, applications and other documents may be selected for the present invention and embodiments thereof.

1. A method of inhibiting neuronal cell death, comprising contacting an HIV-1-exposed neuronal cell with an apoptosis-inhibitory amount of erythropoietin polypeptide, said neuronal cell having been exposed to an HIV-1 virus or protein thereof.

2. The method of claim 1, wherein the amount of neuronal cell death in a neuronal tissue is reduced in the presence of said erythropoietin compared to in its absence.

3. The method of claim 2, wherein neuronal cell death is reduced by 10% in the presence of said erythropoietin compared to in its absence.

4. The method of claim 2, wherein neuronal cell death is reduced by 50% in the presence of said erythropoietin compared to in its absence.

5. The method of claim 2, wherein neuronal cell death is reduced by 100% in the presence of said erythropoietin compared to in its absence.

6. The method of claim 2, wherein neuronal cell death is reduced by 200% in the presence of said erythropoietin compared to in its absence.

7. The method of claim 1, further comprising contacting said neuronal cells with an erythropoietin receptor polypeptide.

8. A method of inhibiting neuronal cell death, comprising preferentially contacting a neuronal tissue with an apoptosis-inhibitory amount of an erythropoietin polypeptide.

9. The method of claim 8, wherein said erythropoietin is administered intranasally.

10. The method of claim 8, wherein said erythropoietin is administered intrathecally.

11. The method of claim 8, further comprising contacting said neuronal tissue with an erythropoietin receptor polypeptide.

12. A method of reducing a symptom a neurological disorder, the method comprising identifying an individual suffering from an infectious disease-associated neuropathy and administering to the individual a therapeutically effective amount of erythropoietin, wherein said infectious disease is HIV-1 infection.

13. The method of claim 12, wherein the neurological disorder is selected from the group consisting of HIV-associated dementia (HAD), minor cognitive/motor disorder (MCMD), neuropathic pain associated with HAD, or neuropathic pain from other causes.

14. The method of claim 12, wherein said individual is a human male.

15. The method of claim 12, wherein said individual is a non-lactating human female.

16. The method of claim 12, wherein the therapeutically effective amount of erythropoietin is between about 1 U/kg/day and 2000 U/kg/day.

17. The method of claim 12, wherein the therapeutically effective amount of soluble erythropoietin is between about 100 U/kg/day and 1500 U/kg/day.

18. The method of claim 12, wherein the therapeutically effective amount of erythropoietin is between about 1000 U/kg/day and 1250 U/kg/day.

19. The method of claim 12, wherein the erythropoietin is administered intranasally.

20. The method of claim 12, wherein the erythropoietin is administered intravenously.

21. The method of claim 12, further comprising administering to the individual a therapeutically effective amount of a soluble erythropoietin receptor.

22. The method of claim 21, wherein the therapeutically effective amount of the soluble erythropoietin receptor is between about 1 U/kg/day and 2000 U/kg/day.

23. The method of claim 21, wherein the therapeutically effective amount of the soluble erythropoietin receptor is between about 100 U/kg/day and 1500 U/kg/day.

24. The method of claim 21, wherein the therapeutically effective amount of the soluble erythropoietin receptor is between about 1000 U/kg/day and 1250 U/kg/day.

25. The method of claim 21, wherein the soluble erythropoietin receptor is administered intranasally.

26. The method of claim 21, wherein the soluble erythropoietin receptor is administered intravenously.

27. The method of claim 21, wherein the soluble erythropoietin receptor increases the stability of erythropoietin when both are administered to a patient.

28. A method of reducing a symptom a neurological disorder, the method comprising identifying an individual suffering from a chronic neuropathy and administering to the individual a therapeutically effective amount of erythropoietin.

29. The method of claim 28, wherein said neuropathy is diabetes-associated neuropathy or neuropathic pain.

30. The method of claim 28, wherein said individual is a human male.

31. The method of claim 28, wherein said individual is a non-lactating human female.

32. The method of claim 28, wherein the therapeutically effective amount of erythropoietin is between about 1 U/kg/day and 2000 U/kg/day.

33. The method of claim 28, wherein the therapeutically effective amount of soluble erythropoietin is between about 100 U/kg/day and 1500 U/kg/day.

34. The method of claim 28, wherein the therapeutically effective amount of erythropoietin is between about 1000 U/kg/day and 1250 U/kg/day.

35. The method of claim 28, wherein the erythropoietin is administered intranasally.

36. The method of claim 28, wherein the erythropoietin is administered intravenously.

37. The method of claim 28, further comprising administering to the individual a therapeutically effective amount of a soluble erythropoietin receptor.

38. The method of claim 37, wherein the therapeutically effective amount of the soluble erythropoietin receptor is between about 1 U/kg/day and 2000 U/kg/day.

39. The method of claim 37, wherein the therapeutically effective amount of the soluble erythropoietin receptor is between about 100 U/kg/day and 1500 U/kg/day.

40. The method of claim 37, wherein the therapeutically effective amount of the soluble erythropoietin receptor is between about 1000 U/kg/day and 1250 U/kg/day.

41. The method of claim 37, wherein the soluble erythropoietin receptor is administered intranasally.

42. The method of claim 37, wherein the soluble erythropoietin receptor is administered intravenously.

43. The method of claim 37, wherein the soluble erythropoietin receptor increases the stability of erythropoietin when both are administered to a patient.

44. A pharmaceutical composition comprising erythropoietin, a soluble erythropoietin receptor, and a pharmaceutically acceptable carrier.

45. A kit comprising in one or more containers, the pharmaceutical composition of claim 44.
